(19) United States
(12) Patent Application Publication
(10) Pub. No.: US 2014/0079662 A1
(43) Pub. Date: Mar. 20, 2014

(54) Compositions and methods for treating respiratory disorders with doxofilline

(71) Applicants: William Wayne Howard, Morristown, NJ (US); Russell Francis Somma, Sparta, NJ (US)

(72) Inventors: William Wayne Howard, Morristown, NJ (US); Russell Francis Somma, Sparta, NJ (US)

(21) Appl. No.: 13/573,431

(22) Filed: Sep. 14, 2012

(51) Int. Cl. A61K 31/787; A61P 11/00; A61P 11/08; A61P 11/06

(52) U.S. Cl. 424/78.13

(57) ABSTRACT

COMPOSITONS AND METHODS FOR TREATING RESPIRATORY DISORDERS WITH DOXOFILLINE.

### BACKGROUND OF THE INVENTION

1. Field of the Invention

The invention is for compositions and methods for treating respiratory conditions in humans with doxofylline.

2. Description of the Related Art

The respiratory conditions that are the subject of this application are asthma, COPD, cystic fibrosis, brochiolitis obliterans, bronchiectasis, emphysema or COPD secondary to antitrypsin deficiency, reactive airway dysfunction syndrome (RADS) and World Trade Center Cough and other conditions that cause impaired lung function due to obstruction of the lung's airway passages. Although the causes and underlying etiology of these disorders are quite different, the treatments for them are quite similar. Two common pharmacological effects sought in all eight disorders are bronchodilation and anti-inflammatory effects. Representative references for this observation include the following. For asthma see Stoloff, 2008 for COPD see Yawn et al, 2012; for Cystic Fibrosis see Colombo, 2003 and Balfour-Lynn, 2009; for bronchiectasis see Martinez-Garcia et al, 2011; for bronchiolitis see Bergeron et al, 2007; for emphysema or COPD secondary to antitrypsin deficiency see Kaplan, 2010; for World Trade Center Cough see Friedman et al, 2006; for reactive airways dysfunction syndrome see Weiden et al, 2010 who note that RADS is primarily an obstructive lung disease and Wedzicha et al, 2012 for treatment of obstructive lung diseases. Wedzicha et al note that long acting beta agonists (LABA) or anti-cholinergic drug are often co-administered with inhaled cortico-steroids with the LABA or anti-cholinergic being a bronchodilator and the steroid acting as an anti-inflammatory agent.

As noted above, a common combination is either a long acting beta agonist or anti-cholinergic combined with a steroid e.g. Advair® (fluticasone and salmeterol) and Symbicort® (budesonide/formoterol fumarate dihydrate). In 2010, Advair was the fourth largest selling pharmaceutical product in the United States with total sales of $3.7 billion (Drug Topics, June, 2011). Furthermore, the dominant route of administration of these drug combinations is inhalation (Lareau and Yawn, 2010). Importantly, inhaled drugs suffer broadly from non-compliance as noted below.

Non-Compliance in Asthma

Compliance with inhaled medication in patients with asthma is known to be poor, with about half of patients under-medicating with respect to current guidelines for optimum disease control. Chatkin et al. (2006) conducted a study in 131 asthma patients in Brazil and found that the overall rate of compliance was only 51.9%. Lacasse et al. (2005) performed a similar study in Canada and concluded that patients took between 48% and 96% of their prescriptions. Finally, Bender et al. (2000) compared four adherence assessment methods: child report, mother report, canister weight, and electronic measurements of metered dose inhaler (MDI) actuation. Participants included 27 children with mild-to-moderate asthma who were followed prospectively for 6 months. All patients used an MDI equipped with an electronic doser attached to their inhaled steroid. At each 2-month follow-up visit, doser and canister weight data were recorded, while child and mother were interviewed separately regarding medication use. They concluded that children and mothers reported, on average, over 80% adherence with the prescribed inhaled steroid. However, canister weight revealed, on average, adherence of 69%, significantly lower than self-report. When adherence recorded by the electronic doser was truncated to no more than 100% of prescribed daily use, average adherence was 50%.

The costs of non-compliance are two-fold: the patient experiences a reduced quality of life and pressure on healthcare systems increases since non-compliant patients may experience worsening of their condition, requiring more costly acute medical interventions. Stern et al. (2006) examined the association between medication compliance and exacerbation in asthmatics patients. This study showed that more compliant patients were significantly less likely to experience an asthma exacerbation than less compliant patients were.

Non-Compliance in COPD

Adherence patterns in COPD do not appear to be different than the details shown above for asthma. Cecere et al 2012 stated that “Adherence to long-acting inhaled medications among patients with COPD is poor.” A second study conducted by Bender et al 2006 used pharmacy records for their analysis. Bender et al stated “This pharmacy database study portrays medication adherence levels to be considerably lower than those reported in most clinical trials, suggesting that most adults taking FCS [fluticasone propionate/salmeterol combination] obtain a single fill before abandoning their controller medication.” A similar set of observations were reported by Lareau and Yawn, 2010: “The rate of >50% poor adherence in the COPD population is not surprising, because people with COPD usually have multiple morbidities and take an average of six medications.” Lareau and Yawn point out that: “Acute and maintenance treatment of COPD relies on inhaled agents to manage and control symptoms and/or complications of the disease, and prevent exacerbations.” In general, it appears that COPD patients take less than 50% of the medication that is need to control their symptoms and a great deal of this non-compliance occurs with inhaled medications.

Non-Compliance in Cystic Fibrosis

The literature on compliance in Cystic Fibrosis is considerably thinner than for asthma or COPD, likely reflecting the relatively small number of patients involved. Marciel et al, 2010 state: “Treatment regimens for patients with cystic fibrosis (CF) are time-consuming and complex, resulting in consistently low adherence rates.”

Oral Dosage Forms Improve Compliance and Health Outcomes Oral drug delivery would provide the benefit of improved compliance in these respiratory disorders. A recent article in the New England Journal of Medicine reported that oral drug compliance is 60% higher than comparable drugs delivered via the inhaled route (Price et al, 2011). Higher compliance is generally associated with improved health outcomes (Richter et al, 2003).

Furthermore, inhaled drugs have been associated with poor oral health such as dental caries, candidiasis, ulceration, gingivitis, periodontitis, halitosis and taste changes (Godara, et al 2011).

Ion Exchange Resins Create Superior Oral Formulations

The use of ion exchange resins (IER) to create oral pharmaceutical formulations can be traced back to the pioneering work of John Keating marked by the issuance of U.S. Pat. No. 2,990,332 to Keating on Jun. 27, 1961. Keating's patent was the first to enumerate the many new and useful applications of IER technology to drug delivery systems.

Over time, a group of benefits associated with the use of IER for pharmaceutical formulations has emerged. These have been enumerated by Bajpai et al 2007 and include improved stability, better dissolution, limiting deliquescence, limiting polymorphism, improving tablet disintegration, improved taste masking and extended release formulations.

It is the object of the present invention to utilize doxofylline in ion exchange resin formulations in oral dosage forms to treat respiratory diseases such as asthma, COPD, cystic fibrosis, brochiolitis obliterans, bronchiectasis, emphysema or COPD secondary to antitrypsin deficiency, reactive airway dysfunction syndrome (RADS), World Trade Center Cough and other conditions that cause impaired lung function due to obstruction of the lung to improve long function via the bronchodilation and anti-inflammatory effects of doxofylline. Further, it is an object of the present invention to use ion exchange resins, to create immediate and extended release oral compositions of doxofylline.

Such a composition of doxofylline has never been revealed in the published literature or issued or pending patents.

The history of prior art for the treatment of respiratory diseases indicates that a serious need exists for a novel and useful treatment that that provides an unexpected advancement in the science of respiratory treatment. For example, the prior art does not provide for xanthine based drugs that have a tolerable side effect profile while delivering the twin benefits of bronchodilation and anti-inflammatory effects to patients in an oral ion exchange resin dosage form. Present treatments must combine a bronchodilator and an anti-inflammatory drug to achieve both effects and the combination is delivered via an inhaled drug route. Theophylline is an oral xanthine but numerous side effects limit its utility. Further, long acting beta agonists (LABAs) and inhaled steroids both have deleterious health effects associated with them (Walters et al, 2007), (Allen, 2005). The inventive step provides for a safer treatment for respiratory diseases that also improves compliance and therefore efficacy by delivering doxofylline in an ion exchange resinate via the oral drug delivery route in either immediate release or extended release formulations.

### SUMMARY OF THE INVENTION

The new method and compositions of this invention use ion exchange resin technology to improve the oral delivery of doxofylline for the treatment of respiratory disorders in humans. The new methods employ compositions that are especially suited to situations wherein patients need either immediate release of doxofylline or extended release of doxofylline in solid or liquid oral forms.

In a first embodiment, the invention is an oral dosage pharmaceutical composition comprising:

a) at least doxofylline bound to an ion exchange resin forming a resinate; said ion exchange resin selected from the group consisting of a weak acid ion exchange resin and a strong acid ion exchange resin and their combination; and

b) optionally one or more pharmaceutically active agents other than doxofylline.

In a second embodiment, the invention is a method of treating a patient comprising oral administration to said patient of an oral dosage pharmaceutical composition comprising:

a) at least doxofylline bound to an ion exchange resin forming a resinate; said ion exchange resin selected from the group consisting of a weak acid ion exchange resin and a strong acid ion exchange resin and their combination; and

b) optionally, one or more pharmaceutically active agents other than doxofylline;

said patient having a condition selected from the group consisting of asthma, COPD, cystic fibrosis, brochiolitis obliterans, bronchiectasis, emphysema or COPD secondary to antitrypsin deficiency, reactive airway dysfunction syndrome (RADS), World Trade Center Cough, and impaired lung function due to obstruction of the lung's airway passages.

### BRIEF DESCRIPTION OF THE DRAWINGS

FIG. 1 is a flow chart that illustrates a process for creating a drug/resin complex, the drug resination process.

FIG. 2 is a flow chart that illustrates a process for adding a coating to a drug resinate to enhance material flow during additional manufacturing processes.

FIG. 3 is a flow chart that illustrates a process for creating an immediate release capsule dosage form containing an immediate release drug resinate.

FIG. 4 is a flow chart that illustrates a process for adding an extended release coating to a drug resinate.

FIG. 5 is a flow chart that illustrates a process for creating a capsule dosage form containing an extended release drug resinate, an immediate release resinate component.

### DETAILED DESCRIPTION OF THE INVENTION

In a first embodiment, the invention is an oral dosage pharmaceutical composition comprising:

a) at least doxofylline bound to an ion exchange resin forming a resinate; said ion exchange resin selected from the group consisting of a weak acid ion exchange resin and a strong acid ion exchange resin and their combination; and

b) optionally one or more pharmaceutically active agents other than doxofylline.

In a second embodiment, the invention is a method of treating a patient comprising oral administration to said patient of an oral dosage pharmaceutical composition comprising:

a) at least doxofylline bound to an ion exchange resin forming a resinate; said ion exchange resin selected from the group consisting of a weak acid ion exchange resin and a strong acid ion exchange resin and their combination; and

b) optionally, one or more pharmaceutically active agents other than doxofylline;

said patient having a condition selected from the group consisting of asthma, COPD, cystic fibrosis, brochiolitis obliterans, bronchiectasis, emphysema or COPD secondary to antitrypsin deficiency, reactive airway dysfunction syndrome (RADS), World Trade Center Cough, and impaired lung function due to obstruction of the lung's airway passages.

By “drug” is meant an active pharmaceutical agent (API) other than food articles that are intended to diagnose, cure, mitigate, treat or prevent disease in man or other animals or that are intended to affect the structure or any function of the body of man or other animals that are physiologically acceptable. The drug could be a combination of active pharmaceutical agents as well as a single agent.

The term “obstructive lung disease” means any disease that causes the airways of the lungs to become narrow or blocked so that a patient cannot exhale completely. Because of damage to the lungs or narrowing of the airways inside the lungs, exhaled air comes out more slowly than normal. At the end of a full exhalation, an abnormally high amount of air may still remain in the lungs.

The term “ion exchange resin” as used herein means any insoluble polymer that can act as an ion exchanger.

Strong acid resins are so named because their chemical behavior is similar to strong acids. During the process of creating the resin polymer, a strong acid such as —SO3H is introduced into the resin. This sulfonic acid group is very highly ionizable and thus produces many ions available for the exchange process during drug resination.

In a weak acid resin the ionizable group introduced to the polymer is a carboxylic acid (—COOH) as opposed to the sulfonic acid group (—SO3H) used in strong acid resins. These resins behave similarly to weak organic acids so are weakly dissociated i.e. have fewer ions available for exchange.

The term “resinate”, or “drug/resin complex” means a complex formed between an active pharmaceutical agent and an ion exchange resin.

A drug/resin complex (resinate) is achieved by an ionic bonding of a drug molecule to a resin bead. For cationic resinates, the drug molecule will only disassociate in the presence of an acidic environment and/or a strong electrolyte solution, e.g. NaCl, both of which are found in the stomach.

The inventive method requires cationic resins as part of the pharmaceutical formulation. The dosage forms can be suspensions, compressed tablets, capsules, granules/multi-particle systems, pellets as well as granules/multi-particle systems, pellets filled into capsules and the like as either immediate release or sustained release formulations or both in one medicament.

The pharmaceutical oral dosage compositions employed in the invention can be formulated, for example, as a suspension, as a capsule or compressed tablet. Furthermore, such compositions may include a first, immediate release (IR) component, and a second, extended release (ER) component.

When the composition includes both such first and second components, by “immediate release” is meant that the release of the pharmacologically active agent from the first component is such that 80%, 85%, 90%, or even 95% of the ultimate extent of release from said component occurs within 45 minutes when dissolution is measured according to the USP 31 NF 26 section 711.

By “extended release” is meant that the pharmaceutically active agent is released from the second component at a controlled rate such that the formulation allows for a reduction in dosing frequency as compared to that presented by a conventional dosage form.

By “pharmaceutically active agent” is meant articles intended for use in the diagnosis, cure, mitigation, treatment, or prevention of disease in man or other animals and articles (other than food) intended to affect the structure or any function of the body of man or other animals that are physiologically acceptable. The agent could be a combination of pharmaceutical active agents as well as a single agent.

By “pharmaceutical composition” is meant the pharmaceutically active agent(s) and other pharmaceutical materials known to those skilled in the art needed to create an oral dosage form.

By “ion exchange resin” is meant an insoluble solid matrix that carries exchangeable ions with either a positive or negative charge. The trapping of ions takes place only with simultaneous releasing of other ions. Ions are exchanged in stoichiometrically equivalent amounts of other ions with the same electrical charge when the ion exchange material is in contact with an electrolytic solution.

The invention comprises both compositions including a single pharmaceutically active agent and fixed combination products containing a second pharmaceutically active agent. Furthermore, the invention comprises compositions including both an IR component and extended release (ER) component. In these embodiments, the IR component (as described above) can also be used as the nucleus to provide the extended release component through the use of an extended release coating.

Ion Exchange Resins

The compositions of the invention include weak and strong acid ion exchange resins. By “weak acid ion exchange resin” is meant in a weak acid resin the ionizable group introduced to the polymer is a carboxylic acid (COOH) as opposed to the sulfonic acid group (SO₃H) used in strong acid resins. These resins behave similarly to weak organic acids so are weakly dissociated i.e. have fewer ions available for exchange.

By “strong acid ion exchange resin” is meant in a strong acid resin the ionizable group introduced to the polymer is a sulfonic acid group (SO₃H) as opposed to the carboxylic acid (COOH) used in weak acid resins. These resins behave similarly to strong organic acids so are strongly dissociated i.e. have many ions available for exchange.

Examples of suitable weak acid ion exchange resins are, for example,

A: Amberlite IRP88 (CAS Registry Number 39394-76-5) manufactured by DOW Chemical

B: DOWEX Mac-3 manufactured by DOW Chemical Examples of suitable strong acid ion exchange resins are, for example,

A: Amberlite IRP69 manufactured by Rohm and Haas

B: Dowex Marathon C, Dowex 88 and Dow XYS-40010 manufactured by Dow Chemical and the like.

### Oral Dosage Delivery Systems

Delivery systems employable in the method of the invention include suspensions, compressed tablets, capsules, granules/multi-particle systems, pellets as well as granules/multi-particle systems, pellets filled into capsules and the like.

### EXAMPLES

Each of the compositions of the examples below are useful for oral administration for respiratory conditions such as asthma, COPD, cystic fibrosis and bronchiectasis.

### Example 1

### Mono-Substance Doxofylline IR Coated Intermediate

| | |
|---|---|
| Doxofylline Resinate Intermediate* | 400 mg |
| HPMC 6 cps | 3 mg |
| Talc | 2 mg |
| Triethyl Citrate | 2 mg |
| Total Dosage Form Weight | 407 mg |

*Equivalent to doxofylline 200 mg.

The process for creating the drug/resin complex and dosage form is as follows.

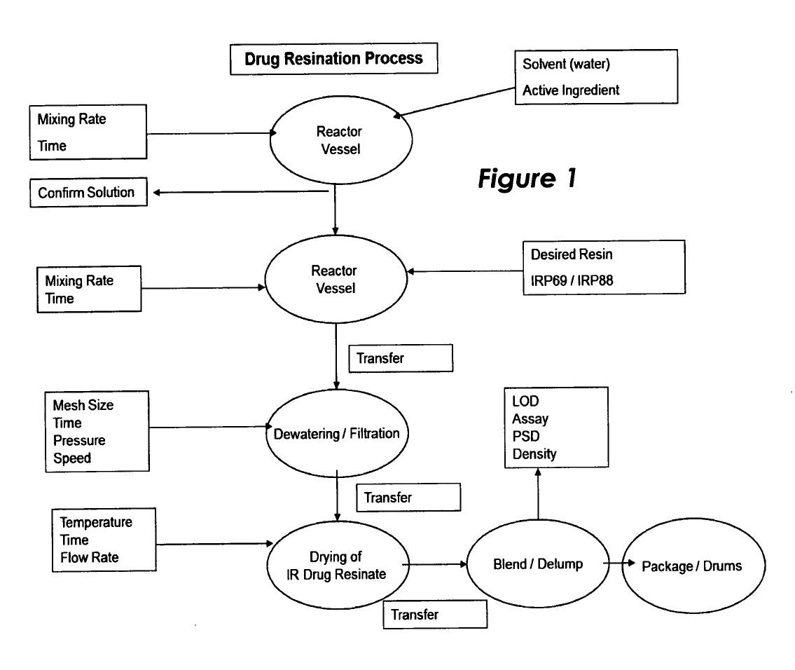

500 mg of IRP-88 from Rohm and Haas (currently DOW) is added to deionized water (2.5 L) which has been heated to 85° C. The resin and water are mixed using a magnetic stirring bar until a uniform suspension is obtained. 150 mg of doxofylline is made into a solution in deionized water and then added to the resin slurry and mixed in the primary vessel with continued mixing for 4.0 hours at 85° C. to create a doxofylline resinate. The slurry is vacuum filtered to separate the resinate from the water. The resin particles are washed three times by re-suspending the particles in 5 liters of deionized water maintained at 85° C. The resulting washed particles are filtered and allowed to cool for 12 hours. This process is repeated in order to generate adequate amounts of the doxofylline resinate to prepare the number of capsules required for dissolution testing. Care is taken during the cooling process to avoid cake formation by periodically mixing the resinate bed with a glass stirring rod. The resinate is then dried using a lab scale fluid bed dryer set at 55° C. inlet temperature. Drying is continued until a residual moisture content of 2.0% or less is obtained. Drug loading is tested and shows approximately 40% drug load or approximately 40 mg of doxofylline per 100 mg of resinate. FIG. 1 is an illustration of the process for creating the resinate intermediate.

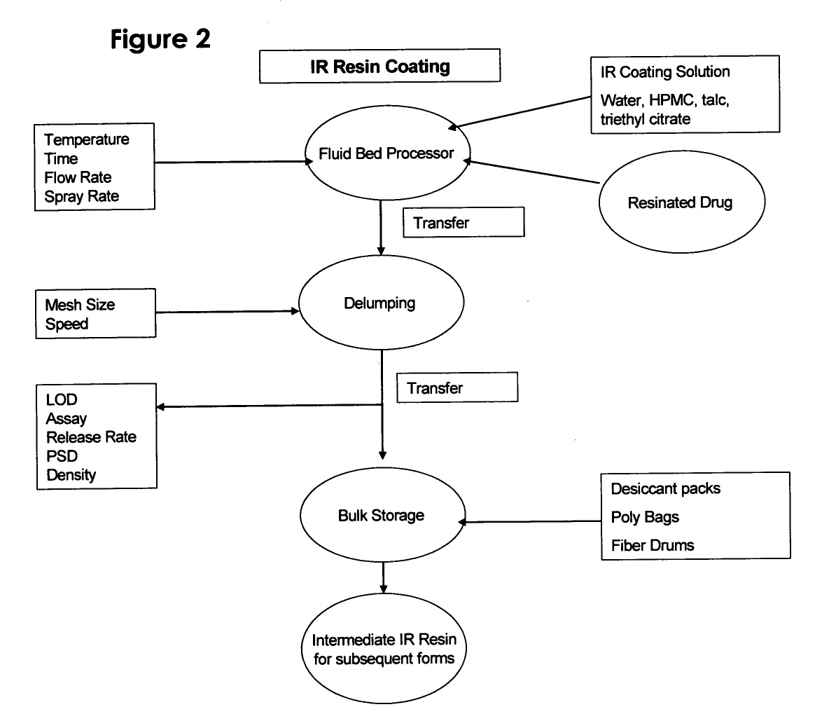

The dried resinate is coated in a lab scale column coater using hydroxypropylmethylcellulose (HPMC) and triethyl citrate as a plasticizer. This is meant to enhance processing and protect the finished resin during subsequent capsule filling. The HPMC coating is a type which has no effect on the dissolution rate and is added as a processing aid to enhance material flow during additional manufacturing steps. This process is illustrated in FIG. 2.

### Example 2

### Formulation for a Commercial Doxofylline Product: Mono-Substance IR Dosage Form, Hard Gelatin Capsule

| | |
|---|---|
| Doxofylline Resinate Coated IR | 400 mg |
| Intermediate* | |
| Magnesium Stearate | 2 mg |
| Colloidal Silicon Dioxide | 3 mg |
| Empty Capsule Shell #3 | 48 mg |
| Total Dosage Form Weight | 453 mg |

*Equivalent to Doxofylline 200 mg.

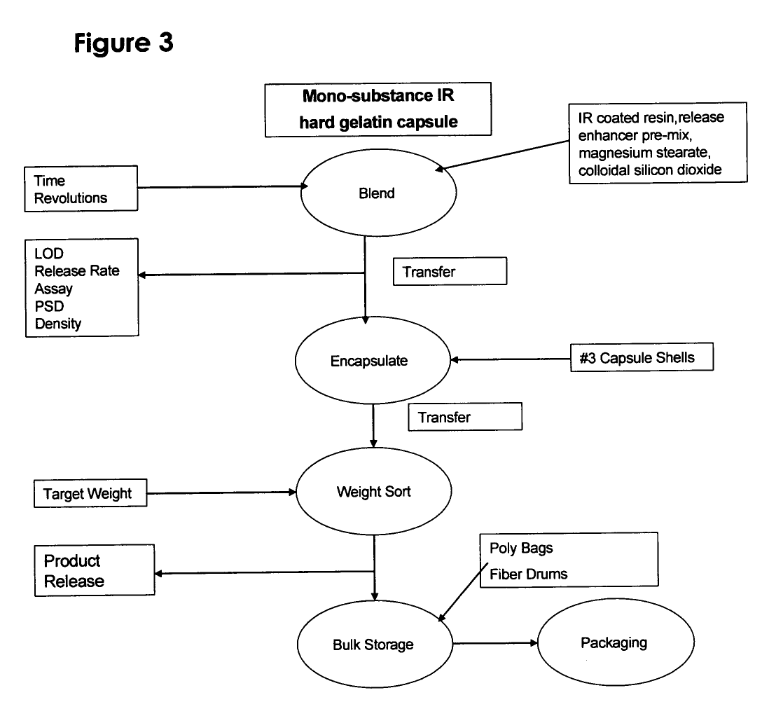

A mono-substance Doxofylline IR dosage form could be prepared as follows. The process begins with the coated Doxofylline resinate as specified in Example 1 and created with the processes described in FIG. 1 and FIG. 2. In the last process step, as shown in FIG. 3, the coated resinate and the other excipients are blended and filled into hard gelatin capsules.

### Example 3

### ER Coated Doxofylline Mono-Substance Component Intermediate for Filling Hard Gelatin Capsules

| | |
|---|---|
| ER Coated Resin Intermediate* | 600 mg |
| Methacrylic Acid Copolymer | 20 mg |
| Talc | 2 mg |
| Triethyl Citrate | 3 mg |
| Colloidal Silicon Dioxide | 3 mg |
| Magnesium Stearate | 2 mg |
| ER component weight* | 630 mg |

*Equivalent to Doxofylline 300 mg

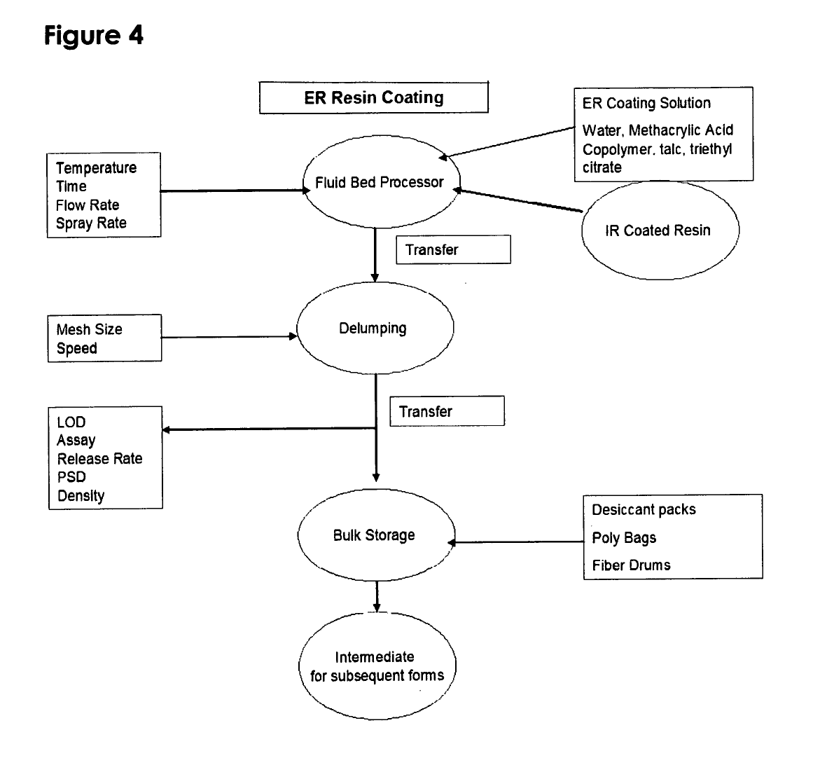

A mono-substance Doxofylline ER intermediate could be prepared as follows. The process begins with the coated Doxofylline resinate as specified in Example 1 and created with the process shown in FIG. 2. Next, the immediate release coated resinate intermediate is further coated as shown in FIG. 4 to create extended release (ER) intermediate particles.

### Example 4

### Formulation for a Commercial Product: IR/ER Doxofylline Encapsulation to Fill a Hard Gelatin Capsule to Deliver 400 mg Doxofylline Total

| | |
|---|---|
| IR component weight* | 200 Mg |
| ER component weight* | 600 Mg |
| Empty Capsule Shell #1 | 76 mg |
| Total Dosage Form Weight | 876 mg |

*Equivalent to Doxofylline 400 mg

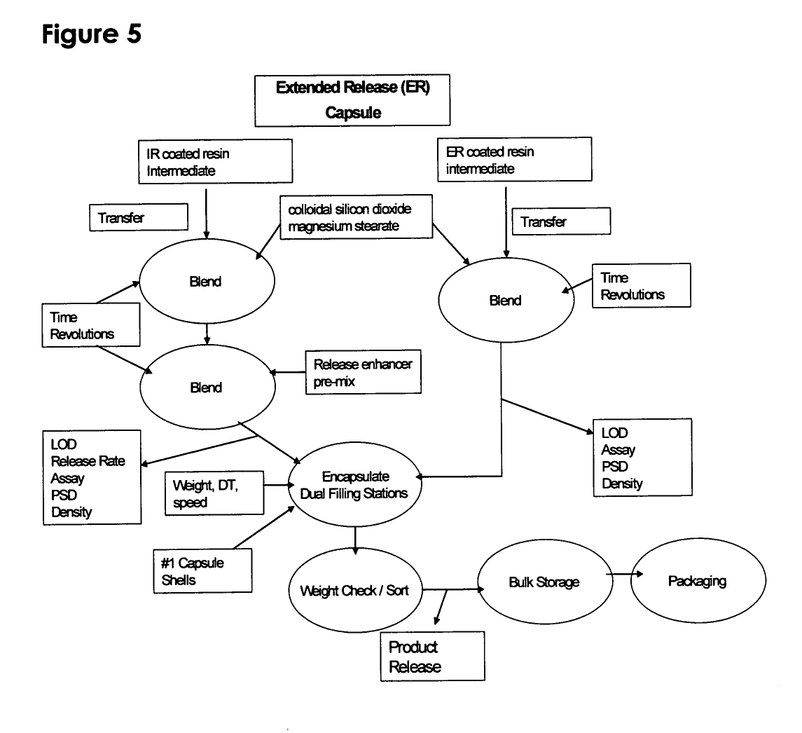

A mono-substance Doxofylline ER dosage form could be prepared as follows. Starting with the Doxofylline IR intermediate specified in Example 1 and the ER intermediate specified in Example 3, the process for filling a capsule to create an extended release capsule (ER) is shown in FIG. 5. Notice that the process uses both the IR and ER coated Doxofylline intermediates from Examples 1 and 3, respectively.

### Formulation Process

One process to manufacture the weak acid IER based products uses a modular approach in the preparation of the required intermediates. This allows maximal utilization of manufacturing capacity. It also assures minimal waste when processing controlled substances which are made to address selected therapeutic applications.

The intermediate materials are used in the manufacture of the final dosage form. The flexibility of the intermediates when combined with commonly used excipients and the release-enhancing agent allows for an array of oral solid dosage forms. Two of the most commonly used in the art are outlined in this process section.

The intermediate process and the dosage form preparation are shown here diagrammatically and fall into the following categories.

### Intermediate Process Categories:

Resination of the Active Ingredient
Immediate Release (IR) Resin Coating
Extended release (ER) Resin Coating

### Dosage Form Process Categories:

Mono Substance IR Hard Gelatin Capsule
Mono Substance IR Compressed Tablet
Extended release Mono Substance Hard Gelatin Capsule

### Other Embodiments

All publications, patent applications, and patents mentioned in this specification are herein incorporated by reference.

Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of medicine, immunology, pharmacology, endocrinology, or related fields are intended to be within the scope of the invention.

### REFERENCES

### Patents

U.S. Pat. No. 2,990,332 Pharmaceutical preparations comprising cation exchange resin adsorption compounds and treatment therewith

### Articles and Books

Allen D B. Inhaled steroids for children: effects on growth, bone, and adrenal function. Endocrinal Metab Clin North Am.2005; 34(3): 555-64.
Bajpai S K, Bajpai M, Saxena S. Ion exchange resins in drug deliver. In Ion Exchange and Solvent Extraction, A Series of Advances, Vol. 18, CRC Press, Boca Raton, 2007.
Balfour-Lynn I M, Welch K. Inhaled corticosteroids for cystic fibrosis. Cochrane Database Syst Rev. 2009 Jan. 2; (1): CD001915.
Bender B, Wamboldt F, O′Connor S, Rand C, Szefler S, Milgrom H and Wamboldt M. Measurement of children's asthma medication adherence by self report, mother report, canister weight and doser ct. Ann Allergy Asthma Immunol, 2000, 85(5): 416-21.
Bender B, Pedan A, Varasteh, L. Adherence and persistence with fluticasone proprionate/salmeterol combination therapy. J Allergy Clin Immunol, 2006, 118(4):899-904.
Bergeron A, Bell A, Chevret S, Ribaud P, Devergie A, Esperou H, Ades L, Gluckman E, Socie G and Tazi A. Combined inhaled steroids and bronchodilators in obstructive airway disease after allogenic stem cell transplantation. Bone Marrow Transplantation, 2007, 39, 547-533.
Cecere Laura M. Cecere, Christopher G. Slatore, Jane E. Uman, Laura E. Evans, Edmunds M. Udris, Chris L. Bryson, and David H. Au Adherence to Long-Acting Inhaled Therapies among Patients with Chronic Obstructive Pulmonary Disease (COPD). 251 COPD, 9:251-258, 2012
Chatkin J, Cavalet-Blanco D, Scaglia N, Tonietto R, Wagner M, Fritscher C. Compliance with maintenance treatment of asthma. J Brass Pneumol. 2006;32(4):277-83.
Colombo C, Russo M C, Za7zeron L, Romano. Liver disease in cystic fibrosis. J. Pediatr. Gastroenterol. Nutr. 43 Suppl 1: S49-55.
Drug Topics, 2010 Top 200 Branded Drugs by Total Prescriptions accessed at http://drugtopics.modernmedicine.com/drugtopics/data/articlestandard//drugtopics/25201 1/727256/article.pdf, Sep. 9, 2012.
Friedman, S, Cone J, Eros-Sarnyai M, Prezant D, Szeinuk J, Clark N, Milek D, Levin S, Gillor. Clinical guidelines for adults exposed to World Trade Center Disaster (Respiratory and Mental Health). City Health Information, NYC Department of Health and Mental Hygiene, September 2006.
Godara N, Godara R, Khullar M. Imapct of inhalation therapy on oral health. Lung India, 2011; 28(4); 272-5.
Kaplan A and Cosentino L. α-Antritrpsin deficiency. Canadian Family Physician, 2010;56:19-24.
Lareau S and Yawn B. Improving adherence with inhaler therapy in COPD. International Journal of COPD, 2010, 5, 401-406.
Lacasse Y, Archibald H, Ernst P, Boulet L. Patterns and determinants of compliance with inhaled steroids in adults with asthma. Can Respir J. 2005, 12(4):211-7.
Marciel K, Salman L, Quittell L, Kawkins M and Quittner A. Cell phone intervention to improve adherence. Pediatr Pulmonol, 2010, 45(2): 157-164.
Martinez-Garcia M, Soler-Cataluna J, Catalan-Serra, P, Roman-Sanchez, P and Tordera M P. Clinical Efficacy and Safety of Budesonide-Formoterol in Non-Cystic Fibrosis Bronchiectasis. Chest, 141, 461-468, 2011.
Page P. Doxofylline: A “Novofylline”. Pulmonary Pharmacology & Therapeutics, 2010, 23:231-234.
Price D, Musgrave S D, Shepstone L, Hillyer E V, Sims E J, Gilbert R F T, Juniper E F, Ayres J G, Kemp L, Blyth A, Edward M A, F. Wilson, M., Wolfe S, Freeman D, Mugford M, Murdoch J, and Harvey I. N. Leukotriene Antagonists as First-Line or Add-on Asthma-Controller Therapy Engl J Med 2011; 364:1695-1707.
Richter A, Anton S, Koch P, and Dennett S. The Impact of Reducing Dose Frequency on Health Outcomes. Clinical Therapeutics (2003); 25(8):23072335.
Stern L, Berman J, Lumry W, Katz L, Want L, Rosenblast L, Doyle J. Medication compliance and disease exacerbation in patients with asthma: a retrospective study of managed care data. Annals of Allergy, Asthma & Immunology. 97(3): 402-408.
Stoloff W. Asthma management and prevention: current perspectives. Clinical Cornerstone, 2008, 9(2), 6-20.
Walters E H, Gibson P G, Laserson, T J, Walters J A. Long-acting beta2-agonists for chronic asthma in adults and children where background therapy contains varied or no inhaled corticosteroid. Cochrane Database Syst Rev., 2007 Jan. 24;(1): CD0011385.
Wedzicha J, Decramer M, Seemungal T. The role of bronchodilatory treatment in the prevention of exacerbations of COPD. European Respiratory Journal, 2012 (Epub ahead of print).
Weiden M, Ether N, Nolan A, Rom W, Comfort A, Gustave J, Zeirg-Owens R, Zheng S, Goldring R, Berger K, Coxenza K, Lee R, Webber M, Kelly K, Aldrich T Prezant D. Obstructive airways disease with air trapping among firefighters exposed to World Trade Center dust. Chest, 2010; 137(3):566-574.
Yawn B, Colice G, Hodder R. Practical aspects of inhaler use in the management of chronic obstructive pulmonary disease in the primary care setting. Int J Chron Obstruct Pulmon Dis, 2012, 7:495-502.

What is claimed is:

1. An oral dosage pharmaceutical composition comprising:
a) at least doxofylline bound to an ion exchange resin forming a resinate; said ion exchange resin selected from the group consisting of a weak acid ion exchange resin and a strong acid ion exchange resin and their combination; and
b) optionally one or more pharmaceutically active agents other than doxofylline.

2. The oral dosage pharmaceutical composition of claim 1 useful or treating an obstructive lung disease in a human patient.

3. The oral dosage pharmaceutical composition of claim 1, wherein said oral dosage pharmaceutical composition is formulated as a member of the group consisting of a capsule, a powder, a thin film, a caplet, a tablet or a suspension.

4. The oral dosage pharmaceutical composition of claim 2 wherein the doxofylline dosage is from about to 50 mg to about 1000 mg.

5. The oral dosage pharmaceutical composition of claim 1, wherein said composition is capable of immediate release of at least one pharmaceutically active agent.

6. The oral dosage pharmaceutical composition of claim 1, wherein said composition is capable of extended release of at least one pharmaceutically active agent.

7. The oral dosage pharmaceutical composition of claim 1, comprising an immediate release component and an extended release component:

8. The oral dosage pharmaceutical composition of claim 1, wherein at least two pharmaceutically active agents are bound to a first ion exchange resin.

9. The oral dosage pharmaceutical composition of claim 1, comprising a first and a second ion exchange resin; wherein said second ion exchange resin is bound to a pharmaceutically active agent and is coated with an extended release coating.

10. The oral dosage pharmaceutical composition of claim 8, wherein said second ion exchange resin is bound to the same pharmaceutically active agent as the first ion exchange resin.

11. A method of treating a patient comprising oral administration to said patient of an oral dosage pharmaceutical composition, said composition comprising:
a) doxofylline only bound to a weak acid ion exchange resin forming a weak acid doxofylline resinate; and
b) optionally one or more pharmaceutically active agents other than doxofylline;
said patient having a condition selected from the group consisting of asthma, COPD, cystic fibrosis, brochiolitis obliterans, bronchiectasis, emphysema or COPD secondary to antitrypsin deficiency, reactive airway dysfunction syndrome (RADS), World Trade Center Cough, and impaired lung function due to obstruction of the lung's airway passages;
said pharmaceutical composition providing both immediate release and extended release of said doxofylline.

12. A method of treating a patient comprising oral administration to said patient of an oral dosage extended release pharmaceutical composition, said oral dosage pharmaceutical composition comprising:
a) at least doxofylline bound to a weak acid ion exchange resin and to a strong acid ion exchange resin forming weak and strong acid doxofylline resinates; and
b) optionally one or more pharmaceutically active agents other than doxofylline;
said patient having a condition selected from the group consisting of asthma, COPD, cystic fibrosis, brochiolitis obliterans, bronchiectasis, emphysema or COPD secondary to antitrypsin deficiency, reactive airway dysfunction syndrome (RADS), World Trade Center Cough, and impaired lung function due to obstruction of the lung's airway passages; and wherein said extended release pharmaceutical composition also provides immediate release of said doxofylline.
